SEQUENCE LISTING (19 sequences)

SEQ ID NO: 1 - DNA, 435 bp, Homo sapiens
ttgccaggttttcaataaagaggaagagaaaggccaccaaatagtttgcttcttaagttgacatagttgtaacagtagtttaaaaactgaaatatttaaaaattcttaatttaaatattatatgtattgactgttaaaaaataaaaaagcctaacagttagcttaaataaaaccacttgaatgtctatgatctctgatatcttgtgtttgcctaaagactgtgatgagaacacgrgtgatgttgatggtaaatggactccctgaggtggagtcagctcactcattggctggatgatgagaccccttagagcagaaagggacagagaggcaatcagcccatgctgcagaaatgtaagaacaccttccactgcatccccagtaaaaatatttttaacccaaaattaatctggaaaacattttcaaaataaattactc

SEQ ID NO: 2 - DNA, 401 bp, Homo sapiens
attccttaatgtttccttctagattcagagcctaaacagcaccattacccagctggccctccccattcttcctaaccaccacccgaagtgttggggacagtctctttttgctcccctccctaccaggacagtgataccctcccaggagggtctaacactatggaacccttgatatcaaggcctgatcttgtcccttccttwgttcttggtgtctggcccactctaagctgtgaaattttcccccatttttgcagctccctgccctggaggaccagctcagcaccctcctagccccggtcatcatctcctccatgacgatgctggagaagctctcggacacctacacctgcttctccacggaaaatggcaacttcctgtatgtccttcacctggtgagtcta

SEQ ID NO: 3 - DNA, 3714 bp, Homo sapiens
ggtcctacccggaagcgcgcccgggctcctgcaggcggggcgctgtgcgcgccgcgatccggtacgtgggcctccgggctgtcccctctgggggcggcgatcctccctccggagccccccttcaaccctcccggaagtgaggaccagggatgctgtgctgctctcccatgagccagtcaccgagtcggtctgctgcagccctttctgaacctctggccgtctggatgctccactgtgcttgccaagatgaagtgcgtcttggtggccactgagggcgcagaggtcctcttctactggacagatcaggagtttgaagagagtctccggctgaagttcgggcagtcagagaatgaggaagaagagctccctgccctggaggaccagctcagcaccctcctagccccggtcatcatctcctccatgacgatgctggagaagctctcggacacctacacctgcttctccacggaaaatggcaacttcctgtatgtccttcacctgtttggagaatgcctgttcattgccatcaatggtgaccacaccgagagcgagggggacctgcggcggaagctgtatgtgctcaagtacctgtttgaagtgcactttgggctggtgactgtggacggtcatcttatccgaaaggagctgcggcccccagacctggcgcagcgtgtccagctgtgggagcacttccagagcctgctgtggacctacagccgcctgcgggagcaggagcagtgcttcgccgtggaggccctggagcgactgattcacccccagctctgtgagctgtgcatagaggcgctggagcggcacgtcatccaggctgtcaacaccagccccgagcggggaggcgaggaggccctgcatgccttcctgctcgtgcactccaagctgctggcattctactctagccacagtgccagctccctgcgcccggccgacctgcttgccctcatcctcctggttcaggacctctaccccagcgagagcacagcagaggacgacattcagccttccccgcggagggcccggagcagccagaacatccccgtgcagcaggcctggagccctcactccacgggcccaactggggggagctctgcagagacggagacagacagcttctccctccctgaggagtacttcacaccagctccttcccctggcgatcagagctcaggtagcaccatctggctggaggggggcaccccccccatggatgcccttcagatagcagaggacaccctccaaacactggttccccactgccctgtgccttccggccccagaaggatcttcctggatgccaacgtgaaggaaagctactgccccctagtgccccacaccatgtactgcctgcccctgtggcagggcatcaacctggtgctcctgaccaggagccccagcgcgcccctggccctggttctgtcccagctgatggatggcttctccatgctggagaagaagctgaaggaagggccggagcccggggcctccctgcgctcccagcccctcgtgggagacctgcgccagaggatggacaagtttgtcaagaatcgaggggcacaggagattcagagcacctggctggagtttaaggccaaggctttctccaaaagtgagcccggatcctcctgggagctgctccaggcatgtgggaagctgaagcggcagctctgcgccatctaccggctgaactttctgaccacagcccccagcaggggaggcccacacctgccccagcacctgcaggaccaagtgcagaggctcatgcgggagaagctgacggactggaaggacttcttgctggtgaagagcaggaggaacatcaccatggtgtcctacctagaagacttcccaggcttggtgcacttcatctatgtggaccgcaccactgggcagatggtggcgccttccctcaactgcagtcaaaagacctcgtcggagttgggcaaggggccgctggctgcctttgtcaaaactaaggtctggtctctgatccagctggcgcgcagatacctgcagaagggctacaccacgctgctgttccaggagggggatttctactgctcctacttcctgtggttcgagaatgacatggggtacaaactccagatgatcgaggtgcccgtcctctccgacgactcagtgcctatcggcatgctgggaggagactactacaggaagctcctgcgctactacagcaagaaccgcccaaccgaggctgtcaggtgctacgagctgctggccctgcacctgtctgtcatccccactgacctgctggtgcagcaggccggccagctggcccggcgcctctgggaggcctcccgtatccccctgctctaggccaaggtggccgcagtctgcctttgcatcctgtcctccagccacccttgcttgccactgttccccatgacgagagcctcctgtctgcagtggccatcctgaggatagggcagagtgcccagggtggccccagggcttctaaaaccccacctagaccaccctccatgtcaggtactgagcaaggccccagatccttctctctggaggaagagggaagcccaggggtcctgtttgtaaaacaacggtggcaacagctcctcttccagagctgcctctgcctttatcctgggagatggggaggaagccccatctctgctgttccctgcgtggaggaagcccacccagcaagctctctcctaccccaggtaaaaggtgctcctttgcctgggtttgaattccagcgctgccacttcctctctgcacctcctggcaagtttcttctattccccacgtttaaagcgatggcacctccgtcccagggtggtgtgaggattacccagtgtggtaggtgctcaataaatgttggtcattgttatcactgaagcccaacatgctagtgcttctagacccttctgtcagtgctgataagcccttgctaagtcccagccccttcatgcttggctggcgtctgccctagggctggggttctcaagcccctggccctggcccagagatttggattcccttggcggccgtggagcccagcctttgatgtctttcaaagcttctgtggtgcgccctggattgagaaccaccacccgaggggtacagcccctctcttccaaccgagaagttcctgtcccagaatggacccagggacaagagaccctgagagccctgggactgggagtgtctgctcctctgaggccaggaggccggtgctgggccagagaggacggcgtggcgaaagtcagcgtccactgcagcacaggatcagatggccgtgtgctgtgcatgcaggagcctcgccttctgtgtctttagtcttgagccaaaatttgctcaaagactgatctcttccttgcagggaacagctttggggctgggggaactagaacccacatgttggtctaaaccctgagaaggtggcagtgaggaagtatcccctcaggtgactggatctgtgttcctccttaacatcatctgatggaatggcaatgaaaagcgtggattgtggaaaatacagaaaaacataaaggaaaaaactccaatcccctgagcccaccactgttcaggacccctgcttttgtcacctactatttccctttagtttttagcagcggctggatgtgatatgtctagtttaaccagtccccttgatctttctatataataaataacacaggagtgaacatcctgaatcag

SEQ ID NO: 4 - DNA, 859 bp, Homo sapiens
  misc_feature (145)..(244): n is a, c, g, or t
gattacaggcggataccaccacgcccaggtaaattttgtatttttagtagagatggggtttcaccatgttagccaggctggtctccaactcctggcctcaagtgatggggtttgagggccggatggaacgaaaacgacattaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttatggcttcttagatgagggagaaccacgtagggatggagaaagcttgggggcagggccagggagcagggcggtaaagcatctggggtactgacacattgtgaattagctackgctgccatgccttaaggtttgcctgaagctgagtggatgtttactgctgtgctgggaagagcagaggccatgtctatggccttcaggggtagggggaagcacacctgatgccaccgtcccctaccctcatacaaccttcttcacatcttctaggggatattgggctgagtctacagcgtgtcttcacagatctgaagaacatggatgccacctggctggacagcctgctgaccccagtccggttgccctccatccaggccattccctgtgcaccgtagcagggcccctgggcccctcttattcctctaggcaagcaggacctggcatcatggtggatatggtgcagagaagctggacttctgtgggcccctcaacagccaagtgtgaccccactgccaagtggggatggggcctccctccttgggtcattgacctctcagggcctggcaggccagtgtctgggtttttcttgtggtgtaaagctggccctgcctcctggg

SEQ ID NO: 5 - DNA, 601 bp, Homo sapiens
acctggaggataatttgctaactttttctataaagccatcatcatattaacggatcctaaaggctgattattgaagcctgatgtgcatttcccgaactagcagggctggggcatgttggggcagaggatgcaggccagggacccatcgctgatagtgcctgactcacagagctgtctgatgccccaaggcttgcttcaggacggcctgtcagaggccaggcctcccacctgccttcccttcccatggtggctttcccaccagtcaagccacgtgaatgtggcacttgtgggacaatgcaarcagccaggtgacaacagcagctacccatcctctgatttggaagcttcactggttctctctcctcactgagaaacggtcacttcaagagtgcccaggtaggaaggggctttaccttcatgatgtcctcaggtaatttcccttcctcatcctcatctgttgtagctgtggatggggaaagcagagaggttggctggcagtcagccacactcaccctgcagttccagttccagcccaccagatccccctgccctttctctgtctctctgtctctctgacacacacacacacacacacaccctc

SEQ ID NO: 6 - DNA, 601 bp, Homo sapiens
tagagattccagaacaggaccctggcctggtgactcagcctctcaaaccctgaagccacgccgcttcccacccctaccctacttccttcctcaccctcagatgctgggctacagaggaggaaggagaaccagcaccccaaaatgcagcccctggcccccttccctcctctcaccagcccccactgtactgcagcccactctgaactgccttcctagtgtccccgtcgcttgcctccccctatggtggctaagactgggcaatgcccaactcaatcaattcagtgccaggtggagttctgakcatcttttctctctcaggaagcccttcacaggacccagacagtcaagcaggcaggccaggccccaggagcaccaaccttcagaggtggagggcatgggtgacacctggaagttgtacagatcactggtgctgtccggcacaacttccactgggatgtgccagtcggggagagtgctgctgacagcacatggcgacagtgctggggaacagcagaagccacaggtcaaggttgtgtgctttcttagtttgcaagacatcgagcgccctccgaccaaccctgcagcctgcactaatgggcca

SEQ ID NO: 7 - DNA, 601 bp, Homo sapiens
cagtggaagaaatgctaaggtgggcctgggcctaagctgctttctccctcgacagtcatgtggggattccagccctgataccttctctgatggactcagcagctccactctgcctgatgaccacagcagctacacagttccaggctacatgcaggacttggaggtggagcaggccctgactccaggtgagctggtccaggtctggcaggagaccccacaggtcagtgggatgactctttctcttggaggcatggtgctggcacatggtggcccattagtgcaggctgcagggttggtcggrgggcgctcgatgtcttgcaaactaagaaagcacacaaccttgacctgtggcttctgctgttccccagcactgtcgccatgtgctgtcagcagcactctccccgactggcacatcccagtggaagttgtgccggacagcaccagtgatctgtacaacttccaggtgtcacccatgccctccacctctgaaggttggtgctcctggggcctggcctgcctgcttgactgtctgggtcctgtgaagggcttcctgagagagaaaagatgatcagaactccacctggcactgaattgattgagt

SEQ ID NO: 8 - DNA, 601 bp, Homo sapiens
agtccagccgagatgctaagagcaaggccaagaggaaggtgagtgtggtcctaagcagccaggcctttggtcacctgtgggccagggtgagcagtggaagaaatgctaaggtgggcctgggcctaagctgctttctccctcgacagtcatgtggggattccagccctgataccttctctgatggactcagcagctccactctgcctgatgaccacagcagctacacagttccaggctacatgcaggacttggaggtggagcaggccctgactccaggtgagctggtccaggtctggcaggrgaccccacaggtcagtgggatgactctttctcttggaggcatggtgctggcacatggtggcccattagtgcaggctgcagggttggtcggagggcgctcgatgtcttgcaaactaagaaagcacacaaccttgacctgtggcttctgctgttccccagcactgtcgccatgtgctgtcagcagcactctccccgactggcacatcccagtggaagttgtgccggacagcaccagtgatctgtacaacttccaggtgtcacccatgccctccacctctgaaggttggtgctcctggggcct

SEQ ID NO: 9 - DNA, 601 bp, Homo sapiens
gctggtggcagacttgtgtttctggagaagagagtcgatcatctcagcaaattctcaaagggaaaagccaagatcttagaaagtgtgtggcttcagggggtttgtggctagatgaaagttctccctggcaaaagcatctgtgaaaagcagctgtaagccagggcactgaaagagacccaggtctgcctttttcttcgtgttgaccaaggcccttggtccaagcctcatgtggttggtggcctcctttatccttgagagatggagctctaggcccatctcagaacagtcagcccacccattyagtaactgttctctgctgcccagtctgtgcccactctaccctctggctgctgatagcccaaggaggaagactgggcatagtctgagacacagatagtacactttggggatatggggactctagtgcttctggctgggcccttcactgaggcccgctagatgtgtttaagccaagcctgggcatttgagaaggcccagggcctaggacctgcagagtgtcaccgggagtacctgctggtttgaccactgtggctctctggtagcataagaggtcaggggtaccttgccttcctccttcagg

SEQ ID NO: 10 - DNA, 701 bp, Homo sapiens
cccatcttgaggctggcttaaacagaccactctggatctctcaggagggacacctagtttggatgagctgcagcattattagctcacaaagacctccctctgcctgttacacatgtgctaggacccacacagggcaccctcccccaaagccctggttttgaagctctgggatgtttctctctggcttgtaagcacccacawggaagtaagaacttcttccattagaaaggactcctcaggacacctgggagcatgggctcttacacagggggtcctggtcataccactgagggagctctgggctagacttggatggtgaacactgtgtaaccctggcattgtcactgtatctctttgcccctcagttttctcttctcggaaatgagaaaacatatccaacaaaattttgaggattaaaaaccagaggatgtgtagggacacagtgacaaatatgaagtctaaggtcttactgttattatatcacttatggttaacagtaaagatttctgagtcagaccgttcaagttcaaatcttggcttcatcactttttgtgtgatcttatgatctacctctcagtgcctctgtttacttatctgaaaatgatgacattagtaagatctaacccacaggactactgcgaggattaaatgacacaatgtaaataacatacttagcaggtgccaggcacacagggagtgtt

SEQ ID NO: 11 - DNA, 790 bp, Homo sapiens
cagcactctgcagggctccaatcgaacaaatagaagactgagaagtggatgctgctgggcagaaacgtgcctggcttagcagaggacaaacgagttaatcttgcaccagtcactctggcccaagaagcctatagctggtgcacttggggcaacatagaccctatagacttagtagcaatgatagtattcataataatagctaatgcttactgaacactccctgtgtgcctggcacctgctaagtatgttatttacattgtgtcatttaatcctcgcagtagtcctgtgggktagatcttactaatgtcatcattttcagataagtaaacagaggcactgagaggtagatcataagatcacacaaaaagtgatgaagccaagatttgaacttgaacggtctgactcagaaatctttactgttaaccataagtgatataataacagtaagaccttagacttcatatttgtcactgtgtccctacacatcctctggtttttaatcctcaaaattttgttggatatgttttctcatttccgagaagagaaaactgaggggcaaagagatacagtgacaatgccagggttacacagtgttcaccatccaagtctagcccagagctccctcagtggtatgaccaggaccccctgtgtaagagcccatgctcccaggtgtcctgaggagtcctttctaatggaagaagttcttacttccatgtgggtgcttacaagccagagagaaacatcccagagcttcaaaaccagggctttgggggagggtgccctgtgtggg

SEQ ID NO: 12 - DNA, 161 bp, Homo sapiens
aaccgggccggaagggttagcgtcctggtcttagcgttgtgggcgctgtggctgtcaggaaggcgtagaatggattcaggsgggcgggagggggctgttcagggtgacggctagccctttgctagctagtggttacaactcaagtcaagggaatttcttct

SEQ ID NO: 13 - DNA, 501 bp, Homo sapiens
actcgccggcgcgcggcgttgcccgggcctccgcgcgggctccggggggcgccggaggagctgcgagccgcgggccgcggcgcggggagggcgggacgcggcgtggaccgcccacccggacgaggctgccggcgcccggcagctttcgcagatctgcgtgcgcgcagccgccaggggcctgtaggtggcccgctatgttcgtcccgcgcatccacacgccgtgccggggaccgagtgtcagcccacgcgtgggcgcccagtgctcccggctttcggcggtcccagctccgcgcccaggcgmcaggttttgggctccctgtgctggtggcaagggctggcttactgcccaggtggctggagggaatcgtgacctacggagactgcgggaagaggcgccacaggtgttccttgggccacttctccagaggaggggaaaccgggccggaagggttagcgtcctggtcttagcgttgtgggcgctgtggctgtcaggaaggcgta

SEQ ID NO: 14 - DNA, 601 bp, Homo sapiens
ggatgaggggacaaacacagtgtgttcagataatggaaatacagtgaaaggttcatgcgttcctgttcatacatttcatttgacttatgtcttacagtttggaaataattttgatagtctaattttacaattaggagagatggagagagattatctctattttacagatgagaaaactgagccccagagagggacagtaacttgctaagatcacatagcaagtggaaaaagcacaataagaacccaggctttcagactcaaatcctgtgttctcttttcatccccctttagtttcatcttycctactgccaagggtagggaagctgtcagggacagaaggttggaatgggaccccaggacaagactgagcagagatttgaatgtggggctgaatgtaggggagctcagaaggctcctgggtggccccgagtgttagggagatcatccgagttagggagatcattccagtgcagaggcaccatcttccccatctacctgggcaaggcaaggaggcccaaggggaggttggggcaacaatagtctggtcctggactatgaaatcacaacccgatacagggaaggaagacccagaagaccaggt

SEQ ID NO: 15 - DNA, 2035 bp, Homo sapiens
cgagccccgccgaaccgaggccacccggagccgtgcccagtccacgccggccgtgcccggcggccttaagaaccaggcaacctctgccttcttccctcttccactcggagtcgcgctccgcgcgccctcactgcagcccctgcgtcgccgggaccctcgcgcgcgaccagccgaatcgctcctgcagcagagccaacatgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtagcagggcccctgggcccctcttattcctctaggcaagcaggacctggcatcatggtggatatggtgcagagaagctggacttctgtgggcccctcaacagccaagtgtgaccccactgccaagtggggatgggcctccctccttgggtcattgacctctcagggcctggcaggccagtgtctgggtttttcttgtggtgtaaagctggccctgcctcctgggaagatgaggttctgagaccagtgtatcaggtcagggacttggacaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaaaattggaaatgtcatctaaccattaagtcatgtgtgaacacataaggacgtgtgtaaatatgtacatttgtctttttataaaaagtaaaattgtt

SEQ ID NO: 16 - DNA, 1466 bp, Homo sapiens
ctcaggaccccactgtggccttcagcctcatcatcagccagtttcctagagaattaggttggttttatgtattgagtaacagcttaaccaataacccactggtcttcgattgcattgctcattgcctttttgtgtataggttctctagacacctccatggaagaaaacctcattgcttaaggtttgtttcaaaaatttctggattcattgctagtattgcataagctcattcattctcccctgagttcgatgaaaaacacccaaattcctctaattctcatgttcctctgtgatattgagacacagcgtccaatagttttccaacggaatagcttttcttacctgggaatgtcccccccagatagttgacactcaggaacagcacggawcaataatggctctgcctctgtctcatcatcttcttggaaaaaatgtgagatgtcacaaagggtctcagaaacacagggtagctccctgtataccctggaaaacaacaacagaatttttactatgaatataaggtaggtgcctgatgatagcataggctgtgcaggaagattttatgttaatagccatagactcaatattttatcttagggaagtcattcctcaggcccctacgactccatctcacctctcagactcccatgactctttcttacatctcattatgttaaatttaactggctctctgtttcccactatatgctgctctttccatcctaggaagcagacgtcagtcagttctcaacatctagcatttgccacaaacattggtttcataataggtcaacaagtatgttgacctatataaccttgctaagaattttagggaaaggatgagattcctaatttgtagtctcccttcatccataattggtgcccgagagaataggaccctaaaatgattgggattgcagggcattagtgagattgggcatgttttataagaacccatggaacagttatctcctcttctcccttctgcctgcaaatggtgagaggggttgcataaagcaacaaaaatgctcacagaaaaagaaaattatggatattgtacacactttcttttcccatcaaggatccttattcagatatggaacatgagagtcctatgctagatccttttctcttcttcatttttgaaggcttggtgctgtcctcctatggctggcaggaatcaagattgaggttaggagtgatggagtgtcctttatgccaagatattcaatggccaatatgacagccactagccacacctgcctatttacatttagttttaaattgttaaatgtgaaaatcagttcctcctttgaagtagccatatttcaagtgctcaaaagccacacgtggctcttgcctgccaccatgtaagacatgcctttgctcctcctttgacttctgccatgatggtgaggcctccccagccacgtgaaactaaaagaatttttctgggtaatggacat

SEQ ID NO: 17 - DNA, 5059 bp, Homo sapiens
ctggttctcaacttcttttgaaataatgttcatagagaaggagggctgtctgagattcgagggaaacaagctctcaggacttccggtcgccatgatggctgtgggcggtaaacgcggttagtgcaagcatctgggccatcttcaatggtaaaaaagatacagtaaagacataaataccacatttgacaaatggaaaaaaaggagtgtccagaaaagagtagcagcagtgaggaagagctgccgagacgggtatacagggagctaccctgtgtttctgagaccctttgtgacatctcacattttttccaagaagatgatgagacagaggcagagccattattgttccgtgctgttcctgagtgtcaactatctgggggggacattcccaggagacatttgctcagaagagaatcaaatagtttcctcttatgcttctaaagtctgttttgagatcgaagaagattataaaaatcgtcagtttctggggcctgaaggaaatgtggatgttgagttgattgataagagcacaaacagatacagcgtttggttccccactgctggctggtatctgtggtcagccacaggcctcggcttcctggtaagggatgaggtcacagtgacgattgcgtttggttcctggagtcagcacctggccctggacctgcagcaccatgaacagtggctggtgggcggccccttgtttgatgtcactgcagagccagaggaggctgtcgccgaaatccacctcccccacttcatctccctccaaggtgaggtggacgtctcctggtttctcgttgcccattttaagaatgaagggatggtcctggagcatccagcccgggtggagcctttctatgctgtcctggaaagccccagcttctctctgatgggcatcctgctgcggatcgccagtgggactcgcctctccatccccatcacttccaacacattgatctattatcacccccaccccgaagatattaagttccacttgtaccttgtccccagcgacgccttgctaacaaaggcgatagatgatgaggaagatcgcttccatggtgtgcgcctgcagacttcgcccccaatggaacccctgaactttggttccagttatattgtgtctaattctgctaacctgaaagtaatgcccaaggagttgaaattgtcctacaggagccctggagaaattcagcacttctcaaaattctatgctgggcagatgaaggaacccattcaacttgagattactgaaaaaagacatgggactttggtgtgggatactgaggtgaagccagtggatctccagcttgtagctgcatcagcccctcctcctttctcaggtgcagcctttgtgaaggagaaccaccggcaactccaagccaggatgggggacctgaaaggggtgctcgatgatctccaggacaatgaggttcttactgagaatgagaaggagctggtggagcaggaaaagacacggcagagcaagaatgaggccttgctgagcatggtggagaagaaaggggacctggccctggacgtgctcttcagaagcattagtgaaagggacccttacctcgtgtcctatcttagacagcagaatttgtaaaatgagtcagttaggtagtctggaagagagaatccagcgttctcattggaaatggataaacagaaatgtgatcattgatttcagtgttcaagacagaagaagactgggtaacatctatcacacaggctttcaggacagacttgtaacctggcatgtacctattgactgtatcctcatgcattttcctcaagaatgtctgaagaaggtagtaatattccttttaaattttttccaaccattgcttgatatatcactattttatccattgacatgattcttgaagacccaggataaaggacatccggataggtgtgtttatgaaggatggggcctggaaaggcaacttttcctgattaatgtgaaaaataattcctatggacactccgtttgaagtatcaccttctcataactaaaagcagaaaagctaacaaaagcttctcagctgaggacactcaaggcatacatgatgacagtcttttttttttttgtatgttaggactttaacactttatctatggctactgttattagaacaatgtaaatgtatttgctgaaagagagcacaaaaatgggagaaaatgcaaacatgagcagaaaatattttcccactggtgtgtagcctgctacaaggagttgttgggttaaatgttcatggtcaactccaaggaatactgagatgaaatgtggtaaatcaactccacagaaccaccaaaaagaaaatgagggtaattcagcttattctgagacagacattcctggcaatgtaccatacaaaaaataagccaactctgacatttggattctaccatagactctgtcattttgtagccatttcagctgtcttttgattaatgttttcgtggcacacatatttccatccttttatgtttaatctgtttaaaacaagttcctagtagacaccatctggttgagtcagttttttttatggtgtattttgaacccattctgatagtctcttttaactggaagatttcaattacttacgttaatgtaattattaatatgttaggatttatcctcagtcagccagtttgttatgtcttttctattctactgttatcacatttgtaccacttaaagtggaatctaggcactttatcaccatttagatcctattaccttttctcatctaggatatagttatcttctacataatctttctgtatcttaaaacccatcaataaattattatatattttctacttttaatcactcagaagatttaaaaaactcatgagaagagtaatctgttatgtttttccagatatttaccatttctgttgctcttccttcattattttccaaatttcgttctgcaaatttccacttcttctgatagacgttttttagttcttttagagtggttctgataggtacagattctcttattttttgcttcctctgaggacatctttttctcaccttcattctcagtgatgttttttgcttgtagtatttttagttgacattgttttctgttcagcagtttccttttagcttccgtatttcctgatgagaaatctgcagtcattcaaattgttgtttccctgtatgtagtgtgtcatttttctgtcagatttcaaggtatttatctttagtttttagccatttcattatgttggggatgagtttccttgttttattccctttggaatttgctccaattcataaatttgcagttttatgtcttttaccaaacttagaggttttcagcctaatttctaaaaatactttttattagcctgattttcatctttataggaaatagtttaagtgatgacaagttccaatagcttatatgcccagaaggccttcaaaataagaattttgaaagaatacagaaaacaaacttttatatccttctcatgtcttctactgtaaaattcatatgctttgctactctaaacctagtttgaaatcaacagtcttgagaatagatgaaaattttgatgaatagtggaattcttttaaatggaaacctcttacatgtgattttccttgccatctagaaataaaccatagtatttatgttgaatcaatcaatattatattttgtttttttcctcctcttctgagactcttattgtggaaatgttagacttttatgttttcctaaatgtccctgatattctacttatttagaacatcttttcattttttccattattctgattgggtaattttaatttgtctattttcaaatttgctggagtgttcacctgttgttgtctgtgtcgtcccactgagtgcattcaccaccttttaaattttggtcactgtatgtatcagttctaaaatttccattttgttctctatattttaaatttcttggcttatattctattttcctgcaaatgtgtcagcatttgcttgtttgagctttttttttttcaagacagggtctcaactctgttacccaggctggagtgcagtggtgcgatctcagctcactgcaacctctgcctcctggttcaagcgattattgtgcctcagcctcctgagtagctgggattacaggcatgcaccaccacagcccagctaattttttgtatttttagtagagacagagttttgctatgttggccaggctggttttgaactcctggcctcaagtgatccacccacctcagcctcccaaagtgctgggattacaggccactacacctggcacatttgagtattttttttttttttttttttttgagatggagtctcgctctgtcatctaggctggagtgcagtggtgtgatctcagctcactgcagcctctgtctcccgggctcaagcgattctcttgcctcagcctcctgagtagctaggactacaggtgcatgccaacacgcccggctaatttttttaaaaaatatttttagtagagacagggtttcaccattttggccaggatggtctcgatctcctgacctcatgatccacccgcctcggccttccaaagtgctgggattacaggcatgagccaccgtgcctggcctcatttgagtatttttataatgtctcttttaaagtctttgtcagataattccactgtacatgttattcagtgtttggtgtccactgagttgtcatttgccagacaagtggagatttttgcagctcatccttgtattctcagtagttccgatatgtaccctcgacatgtgaatgttatcttatgagactctgttttatttgtatccaacagaagatgtttattatttatttggctttctgtgaactgaggtcttaatatcagctcattttaaaagtctttgcagtggtattcggatctatcctgtgtgtgcctatgagattgggtgcagtgtatcctgttagctccattctcagggcgtttgaatgtgaattaggaccagcgcaatgaatgctcaagttggggttgggcgttagaattcataaaagtctttatatgctcag

SEQ ID NO: 18 - DNA, 964 bp, Homo sapiens
gcctccggagccgggtgccagcaggcaggctgccattggtcagggccttcagctggtttcctgccaggtcgaggacttccagtttgggcaggaagtggaggctccaccacttaaagaaggccaggtaattgtcacggagacgcagcacctgtaggctcttggggaggttgcgcagggtttggggcaggagggtgtgcaggcggttctgggacaagtccagccagatcaaaccgctcaggccttggaagaagtgcagatagaggtctccctcggcccacatatggcccagtgcattgccgctgaagtccagggcccgcagcgacgtactgcagagctgctgggacacttggctgtggatgttgttgtgggccaggctgaggtggcgcagggtgcgcaggtgagccacgaagctgaagttgtggcccacgccctgcatgccaaagggctggctgttgtagctgaggtccagggcctccagtcgyggtagctccgtgaatgagtgctcgtggtagaggtccagcttattgtgggacaggtctagcacctgcagaccggtcagcggcaggaactgggagccattgactgcctgcgagatgcagttgtggctcaggcgcaggcactgcaggtgcgagagctgggcaaacatctccggctgcacggtcaccaggttgttccgtgacagatccaaggtgaagttgagggtgctgcagttgggcctgaagtcttcagagctgggagtgtccactggggccggagcaaggtccccaggctgcagccagaccttctcccctccatctgcctcccccatggtggctgtcagctccgaagctccgctgatgcggttgtccgacaggtccacgtagcgcaggccagggaaggccctgaagatgccgagctgggcctggttgatgaagttcatctgcagacgcagagtctggagcatgggcaggcgggccagtggccg

SEQ ID NO: 19 - DNA, 3868 bp, Homo sapiens
ggaggtcttgtttccggaagatgttgcaaggctgtggtgaaggcaggtgcagcctagcctcctgctcaagctacaccctggccctccacgcatgaggccctgcagaactctggagatggtgcctacaagggcagaaaaggacaagtcggcagccgctgtcctgagggcaccagctgtggtgcaggagccaagacctgagggtggaagtgtcctcttagaatggggagtgcccagcaaggtgtacccgctactggtgctatccagaattcccatctctccctgctctctgcctgagctctgggccttagctcctccctgggcttggtagaggacaggtgtgaggccctcatgggatgtaggctgtctgagaggggagtggaaagaggaaggggtgaaggagctgtctgccatttgactatgcaaatggcctttgactcatgggaccctgtcctcctcactgggggcagggtggagtggagggggagctactaggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccacgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctatgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagcaggcactcaataaatgctaccgaaggc

(19) United States
(12) Patent Application Publication
(10) Pub. No.: US 2010/0184050 A1
(43) Pub. Date: Jul. 22, 2010

(54) DIAGNOSIS AND TREATMENT OF INFLAMMATORY BOWEL DISEASE IN THE PUERTO RICAN POPULATION

(75) Inventors: Jerome I. Rotter, Los Angeles, CA (US); Kent D. Taylor, Ventura, CA (US); Esther A. Torres, San Juan, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/597,710

(22) PCT Filed: Apr. 25, 2008
(86) PCT No.: PCT/US08/61652

§ 371 Date: Jan. 12, 2010

Related U.S. Application Data
(60) Provisional application No. 60/914,120, filed on Apr. 26, 2007.

(51) Int. Cl. C12Q 1/68

(52) U.S. Cl. 435/6

(57) ABSTRACT

This invention provides methods of diagnosis and treatment of inflammatory bowel disease. In one embodiment, the invention provides methods of diagnosing and/or predicting susceptibility for inflammatory bowel disease in the Puerto Rican population by determining the presence or absence of a risk variant at the HPS1 locus. In another embodiment, the invention further provides methods of diagnosing and/or predicting protection against inflammatory bowel disease by determining the presence or absence of a protective variant at the IRF1 locus. In another embodiment, the presence in an individual of a risk variant at the CARD8 locus is diagnostic of susceptibility to Crohn's Disease in a Puerto Rican individual. In another embodiment, the presence of a risk variant at the TLR-9 locus in an individual is diagnostic of susceptibility to Crohn's Disease.

### GOVERNMENT RIGHTS

This invention was made with U.S. Government support on behalf of the National Institute of Diabetes and Digestive and Kidney Diseases (NIDDK) Inflammatory Bowel Disease Genetics Consortium (IBDGC). The U.S. Government may have certain rights in this invention.

### FIELD OF THE INVENTION

The invention relates generally to the fields of inflammation and autoimmunity and autoimmune disease and, more specifically, to genetic methods for diagnosing and treating inflammatory bowel disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments provide methods for evaluating the likelihood of an individual to have or develop inflammatory bowel disease, comprising determining the presence or absence of a first risk variant at the HPS1 locus, the presence or absence of a second risk variant at the CARD8 locus, and the presence or absence of a third risk variant at the TLR-9 locus, where the presence of one or more risk variants is predictive of inflammatory bowel disease. In another embodiment, the first risk variant at the HPS1 locus comprises SEQ. ID. NO.: 1. In another embodiment, the second risk variant at the CARD8 locus comprises SEQ. ID. NO.: 16. In another embodiment, the third risk variant at the TLR-9 locus comprises SEQ. ID. NO.: 18. In another embodiment, the individual is Puerto Rican.

Other embodiments provide methods of diagnosing susceptibility to inflammatory bowel disease in an individual, comprising determining the presence or absence of a risk haplotype at the HPS1 locus in the individual, where the presence of the risk haplotype is diagnostic of susceptibility to inflammatory bowel disease. In another embodiment, the individual has not been diagnosed with Hermansky-Pudlak Syndrome. In another embodiment, the risk haplotype at the HPS1 locus comprises haplotype block 3. In another embodiment, the risk haplotype at the HPS1 locus comprises SEQ. ID. NO.: 1. In another embodiment, the individual is Puerto Rican.

Other embodiments provide methods of determining a low probability relative to a healthy individual of developing inflammatory bowel disease in an individual, the method method comprising determining the presence or absence of a protective haplotype at the IRF1 locus, where the presence of the protective haplotype at the IRF1 locus is diagnostic of a low probability relative to a healthy individual of developing inflammatory bowel disease. In another embodiment, the protective haplotype at the IRF1 locus comprises H3. In another embodiment, the protective haplotype at the IRF1 locus comprises one or more variant alleles selected from the group consisting of SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13 and SEQ. ID. NO.: 14. In another embodiment, the individual is Puerto Rican.

Various embodiments include methods of diagnosing susceptibility to Crohn's Disease in a Puerto Rican individual, comprising determining the presence or absence of a risk variant at the CARD8 locus, where the presence of the risk variant at the CARD8 locus is diagnostic of susceptibility to Crohn's Disease. In other embodiments, the risk variant at the CARD8 locus comprises SEQ. ID. NO.: 16. In other embodiments, the individual is Puerto Rican.

Other embodiments include methods of diagnosing susceptibility to Crohn's Disease in an individual, comprising determining the presence or absence of a risk variant at the TLR-9 locus, where the presence of the risk variant at the TLR-9 locus is diagnostic of susceptibility to Crohn's Disease. In other embodiments, the risk variant at the TLR-9 locus comprises SEQ. ID. NO.: 18. In other embodiments, the individual is Puerto Rican.

Other embodiments provide methods of treating a non-Hermansky Pudlak form of inflammatory bowel disease in an individual, comprising determining the presence of haplotype block 3 at the HPS1 locus to diagnose the non-Hermansky Pudlak form of inflammatory bowel disease, and treating the non-Hermansky Pudlak form of inflammatory bowel disease. In other embodiments, the individual is Puerto Rican.

Other embodiments provide methods of treating Crohn's Disease in an individual, comprising determining the presence of a risk variant at the CARD8 locus and/or TLR-9 locus, and treating the Crohn's Disease. In other embodiments, the individual is Puerto Rican.

Other features and advantages of the invention will become apparent from the following detailed description, taken in conjunction with the accompanying drawing, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts associations examined between the HPS1 gene and Inflammatory Bowel Disease in a sample from the Puerto Rican population.

FIG. 2 depicts the HPS1 block structure, describing HPS1 Block 1, 2, and 3, with matching markers.

FIG. 3 depicts the IRF1 block structure and associations. The circled sequence of Block 1 describes H3 spanning the IRF1 gene with its corresponding frequency of associations.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“SNP” as used herein means single nucleotide polymorphism.

“Haplotype” as used herein refers to a set of single nucleotide polymorphisms (SNPs) on a gene or chromatid that are statistically associated.

“Risk variant” as used herein refers to an allele whose presence is associated with an increase in susceptibility to an inflammatory bowel disease, including but not limited to Crohn's Disease and ulcerative colitis, relative to an individual who does not have the risk variant.

“Protective variant” as used herein refers to an allele whose presence is associated with a low probability relative to a healthy individual of developing inflammatory bowel disease.

“Risk haplotype” as used herein refers to a haplotype whose presence is associated with an increase in susceptibility to an inflammatory bowel disease, relative to an individual who does not have the risk haplotype.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, the term “HPS” means hermansky-pudlak syndrome. HPS is a rare disease associated with decreased pigmentation, bleeding problems due to platelet abnormality, and storage of an abnormal fat-protein compound. A “non-HPS form of inflammatory bowel disease” is a subtype inflammatory bowel disease where the patient does not have symptoms associated with HPS.

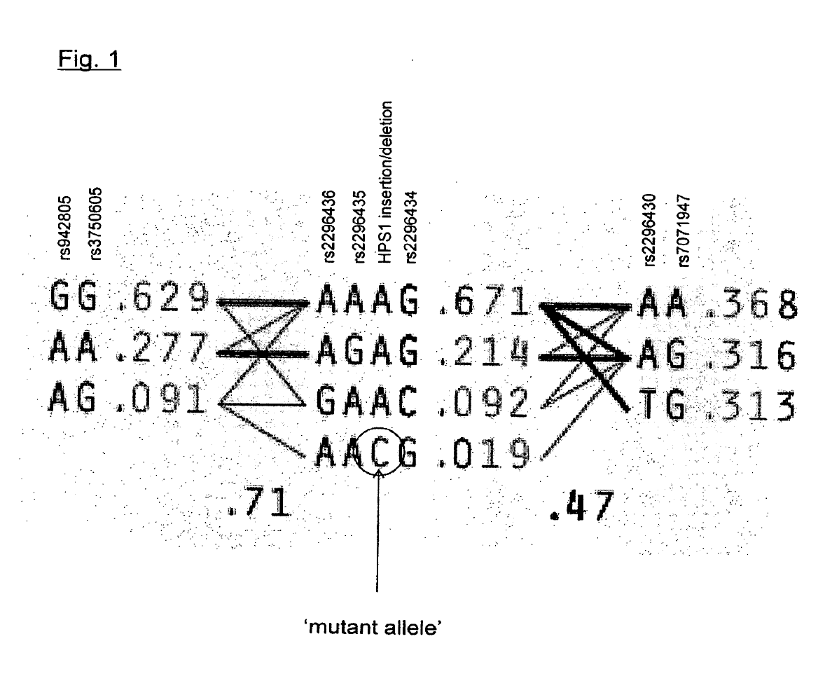

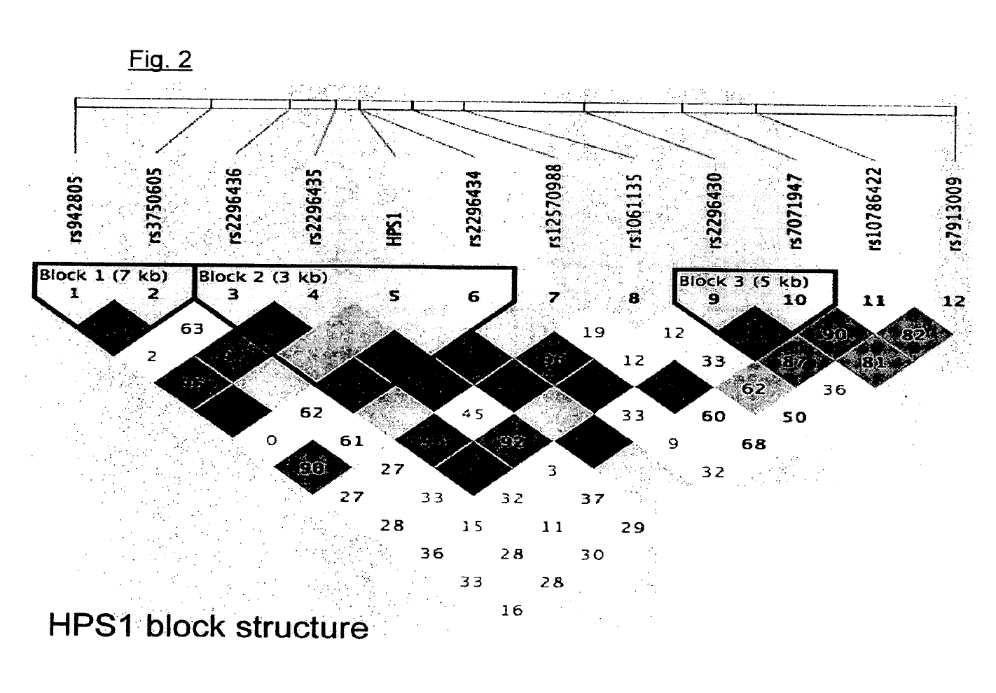

An example of HPS1 is described herein as SEQ. ID. NO.: 3. Block 3 of HPS1 may be identified by SNP rs7071947, also described herein as SEQ. ID. NO.: 1, and/or SNP rs2296430, also described herein as SEQ. ID. NO.: 2. HPS1 and SNPs at the HPS1 locus are also described in FIGS. 1 and 2.

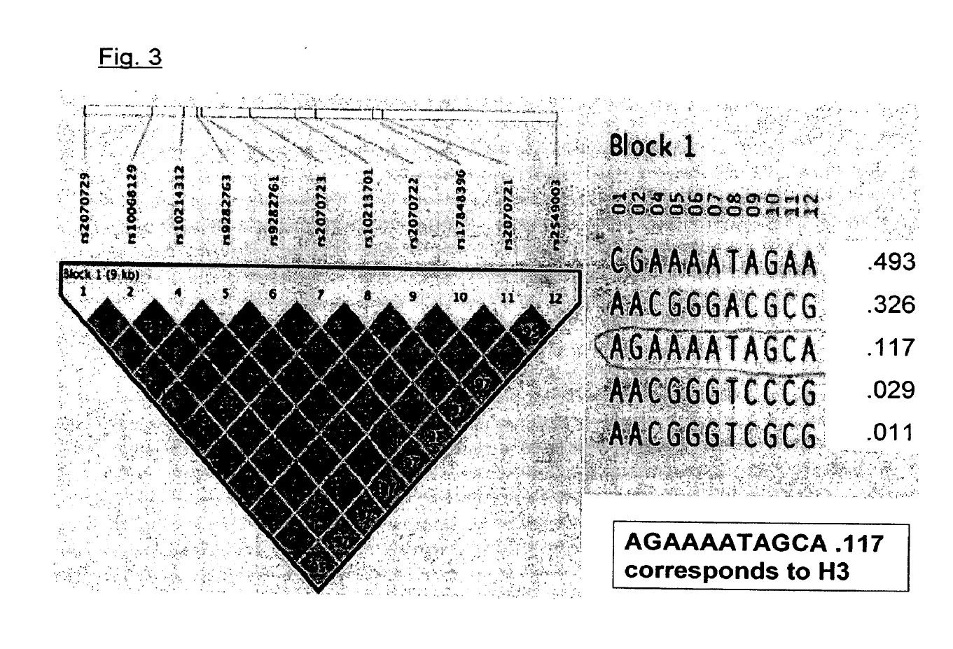

An example of IRF1 is described herein as SEQ. ID. NO.: 15. As used herein, Haplotype H3 of IRF1 is also described as “H3.” H3 may be identified by the alleles of A, G, A, A, A, A, T, A, G, C and A, corresponding to NCBI ID numbers rs2070729, rs10068129, rs10214312, rs9282763, rs9282761, rs2070723, rs10213701, rs2070722, rs17848396, rs2070721, and rs2549003, respectively. NCBI ID numbers rs2070729, rs10068129, rs10214312, rs9282763, rs9282761, rs2070723, rs10213701, rs2070722, rs17848396, rs2070721, and rs2549003, are also described herein as SEQ. ID. NOS.: 4-14, respectively. IRF1 and H3 are also described in FIG. 3.

An example of CARD8 is described herein as SEQ. ID. NO.: 17. SNP 23192A/T at codon 10 of CARD8 is also described herein as SEQ. ID. NO.: 16.

An example of TLR-9 is described herein as SEQ. ID. NO.: 19. SNP 2848A/G of TLR-9 is also described herein as SEQ. ID. NO.: 18.

As used herein, SNP8 is also known as R702W, and R675W. The NCBI SNP ID number for R702W, and R675W, and SNP8, is rs2066844.

As used herein, SNP12 is also known as G88IR, and G908R. The NCBI SNP ID number for G881R, and G908R, and SNP12, is rs2066845.

As used herein, SNP13 is also known as 2936insC, 980fs98IX, frameshift, 3020insC, and 1007fs. The NCBI SNP ID number for 980fs98IX, frameshift, 3020insC, and 1007fs, is rs2066847.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease. Other embodiments provide for methods of treating inflammatory bowel disease including but not limited to Crohn's Disease.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease, as described herein. The methods may also further include recording whether a genetic risk, susceptibility for inflammatory bowel disease including but not limited to Crohn's Disease exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

### I. HPS1

As disclosed herein, inventors examined the association between the HPS1 gene and IBD in a sample from the Puerto Rican population. The inventors examined the DNA of 158 Crohn's Disease patients, 96 ulcerative colitis patients, and 209 ethnically matched controls. Disease was ascertained using standard criteria. SNPs in the HPS1 gene were selected from HapMap data to tag major Caucasian- and African-American haplotypes and were genotyped using Illumina Bead technology. The 14bp insertion was genotyped using ABI microsatellite technology. The association between SNP allele and disease was tested using chi-square. Haplotypes were examined using Haploview.

As further disclosed herein, there is no association between non-HPS-IBD and the HPS1 insertion mutation specific to the Puerto Rican population. The haplotype structure revealed by Haploview analysis shows 3 haplotype blocks, with Block 2 spanning the HPS1 insertion mutation, along with 4 SNPs not in blocks. A major haplotype in Block 3 is tagged by SNP rs7071947. This SNP, not in linkage disequilibrium with the HPS1 mutation, is in fact associated with IBD, particularly in heterozygotes (genotype AA 13% in IBD patients, 20% in controls, genotype AG was 50% in IBD patients, 33% in controls and genotype GG was 37% in IBD patients, 47% in controls, p=0.0019).

As used herein, haplotype block 1, 2, and 3 are described in FIG. 2.

In one embodiment, the present invention provides methods of diagnosing and/or predicting susceptibility for inflammatory bowel disease in an individual by determining the presence or absence in the individual of a risk haplotype at the HPS1 locus. In another embodiment, the risk haplotype comprises block 3. In another embodiment, the risk haplotype comprises SNP rs7071947 variant is diagnostic or predictive of susceptibility to Crohn's Disease. In another embodiment, the individual is Puerto Rican.

In one embodiment, the present invention provides a method of treating non-HPS inflammatory bowel disease by determining the presence of a risk haplotype at the HPS1 locus and treating the non-HPS inflammatory bowel disease. In another embodiment, the individual is Puerto Rican.

### II. IRF1

As disclosed herein, from the Puerto Rican population, the inventors examined DNA from 158 Crohn's Disease patients, 96 ulcerative colitis patients, and 209 ethnically matched controls. Disease was ascertained using standard criteria. SNPs in the IRF1 gene were selected from HapMap data to tag major Caucasian- and African-American haplotypes and were genotyped using Illumina Bead technology. The association between SNP allele and disease was tested using chi-square. Haplotypes were examined using Haploview.

As further disclosed herein, there is no association between IBD and two previously associated variants in the SLC22A4 and SLC22A5 genes in the Puerto Rican population. In contrast, haplotype 3 (H3) of a haplotype block spanning the IRF1 gene is found to be protective for IBD (H3 present in 10% of IBD cases, 19% of controls, p=0.018, pempirical=0.045).

As used herein, H3 is described in FIG. 3.

In one embodiment, the present invention provides methods of diagnosing and/or predicting protection against inflammatory bowel disease in an individual by determining the presence or absence in the individual of a protective variant at the IRF1 locus. In another embodiment, the individual is Puerto Rican.

### III. CARD8

As disclosed herein, the inventors also investigated the association between CD and CARD8 variant in Puerto Rican (PR) population. 38 trio families with one affected offspring, 128 unrelated CD cases and 110 healthy controls were ascertained from Puerto Rico (PR). The SNP (23192A/T) at codon 10 in CARD8 was genotyped using the TaqMan MGB platform (ABI). The transmission disequilibrium test (TDT) was employed to test association with CD using Haploview 3.2. Multiple logistic regression was carried out to analyze the case-control sample.

As further disclosed herein, there is significant distortion of transmission of the CARD8 A allele, the common allele, in CD parent-offspring trios (T: U=22:9, P=0.02). The A allele has a higher frequency in cases than in controls (77% vs 69%, p=0.05). Multivariable analysis shows that the A allele is associated with increased likelihood of CD and there is a dose-response effect (AA vs TT: OR 3.3 p=0.04, AT vs TT: OR 1.9 p=0.8; P for trend=0.03). There is a CARD8 association with CD in the Hispanic population. CARD8, like other CARD family proteins, is involved in apoptosis and NFKB activation. The data shows the existence of a genetic basis for alteration in the innate immune response pathway in the pathogenesis of CD.

In one embodiment, the present invention provides methods of diagnosing and/or predicting susceptibility to inflammatory bowel disease in an individual by determining the presence or absence in the individual of a risk variant at the CARD8 locus. In another embodiment, the risk variant comprises SNP 23192A at codon 10 at the CARD8 locus. In another embodiment, the individual is Puerto Rican.

In one embodiment, the present invention provides a method of treating Crohn's Disease by determining the presence of a risk variant at the CARD8 locus, and treating the Crohn's Disease. In another embodiment, the individual is Puerto Rican.

### IV. TLR-9 and NOD2/CARD15

As disclosed herein, the inventors evaluated the association of CARD15 and other innate immune genes including TLR-9 with CD in Puerto Ricans and describe possible phenotypic associations within CD patients. Puerto Rican CD patients (n=113) were recruited from the University of Puerto Rico IBD Clinic. Ethnically matched controls (n=107) were recruited from patients' spouse or general population. Three variants in CARD15 gene (SNPs 8, 12, 13) and two variants in TLR 9-(2848 A/G, 1237C/T) were genotyped by TaqMan. These polymorphisms were evaluated for their association with CD as well as disease behavior, location and IBD-related surgery. The presence of at least one CARD15 variant was observed in 18.7% of CD as compared to 9.4% of controls (p=0.049). The presence of any CARD15 mutation was positively associated with small bowel disease (p=0.06) and negatively associated with perianal involvement (4% vs 34.7%, P=0.0001). A allele of TLR9-2848A/G was more frequent in subjects with CD-related surgery than those without surgery (54% vs 35%, p=0.007).

As further disclosed herein, the inventors found CARD15 to be more prevalent in Puerto Ricans with CD as compared to ethnically matched controls. The association of variants of both CARD15 and TLR-9 with specific disease behavior or location shows the influence of genetic variants on clinical expression of the disease.

In one embodiment, the present invention provides a method of diagnosing and/or predicting susceptibility to inflammatory bowel disease in an individual by determining the presence or absence in the individual of a risk variant at the TLR-9 locus. In another embodiment, the present invention provides a method of determining whether a patient has an increased likelihood of requiring Crohn's Disease related surgery by determining the presence or absence of a risk variant at the TLR-9 locus. In another embodiment, the risk variant comprises SNP 2848A. In another embodiment, the individual is Puerto Rican.

In one embodiment, the present invention provides a method of treating Crohn's Disease in an individual by determining the presence of a risk variant at the TLR-9 locus and treating the Crohn's Disease. In another embodiment, the individual is Puerto Rican.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a genetic variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

HPS1

The inventors examined the association between the HPS1 gene and IBD in a sample from the Puerto Rican population; that is, to test the possibility as to whether general, non-HPS associated IBD in the Puerto Rican population is due in part to heterozygosity for the known HPS1 mutation. The study examined the DNA of 158 Crohn's Disease patients, 96 ulcerative colitis patients, and 209 ethnically matched controls. Disease was ascertained using standard criteria. SNPs in the HPS1 gene were selected from HapMap data to tag major Caucasian- and African-American haplotypes and were genotyped using Illumina Bead technology. The 14bp insertion was genotyped using ABI microsatellite technology. The association between SNP allele and disease was tested using chi-square. Haplotypes were examined using Haploview.

The inventors found no association between non-HPS-IBD and the HPS1 insertion mutation specific to the Puerto Rican population. The haplotype structure revealed by Haploview analysis is complicated: there are 3 haplotype blocks, with Block 2 spanning the HPS1 insertion mutation, along with 4 SNPs not in blocks. A major haplotype in Block 3 is tagged by SNP rs7071947. This SNP, not in linkage disequilibrium with the HPS1 mutation, is associated with IBD, particularly in heterozygotes (genotype AA 13% in IBD patients, 20% in controls, genotype AG was 50% in IBD patients, 33% in controls and genotype GG was 37% in IBD patients, 47% in controls, p=0.0019).

A SNP in HPS1, but not the Puerto Rican-specific insertion mutation, is associated with non-HPS-IBD in a sample from Puerto Rico. This means that two different independent variations in the same gene, one of which predisposes to a Mendelian disorder (HPS) with IBD, and one which predisposes to non-HPS-IBD, is increased in the Puerto Rican population. This finding shows that selection is acting on the HPS1 gene in Puerto Rico.

### Example 2

### IRF1

The inventors examined the association of SNPs related to the IBD5 locus in the Puerto Rican population, in order to determine if this population, with its own linkage disequilibrium pattern, will aid in distinguishing the responsible gene(s) in this locus. The study examined DNA from 158 Crohn's Disease patients, 96 ulcerative colitis patients, and 209 ethnically matched controls. Disease was ascertained using standard criteria. SNPs in the IRF1 gene were selected from HapMap data to tag major Caucasian- and African-American haplotypes and were genotyped using Illumina Bead technology. The association between SNP allele and disease was tested using chi-square. Haplotypes were examined using Haploview.

The inventors found no association between IBD and two previously associated variants in the SLC22A4 and SLC22A5 genes in the Puerto Rican population. In contrast, haplotype 3 (H3) of a haplotype block spanning the IRF1 gene is found to be protective for IBD (H3 present in 10% of IBD cases, 19% of controls, p=0.018, pempirical=0.045). IRF1, rather than SLC22A4 or SLC22A5, is important for IBD susceptibility in the Puerto Rican population.

### Example 3

### CARD8

The inventors also investigated the association between CD and CARD8 variant in Puerto Rican (PR) population. 38 trio families with one affected offspring, 128 unrelated CD cases and 110 healthy controls were ascertained from Puerto Rico (PR). The SNP (23192A/T) at codon 10 in CARD8 was genotyped using the TaqMan MGB platform (ABI). The transmission disequilibrium test (TDT) was employed to test association with CD using Haploview 3.2. Multiple logistic regression was carried out to analyze the case-control sample.

The inventors found significant distortion of transmission of the CARD8 A allele, the common allele, in CD parent-offspring trios (T: U=22:9, P=0.02). The A allele has a higher frequency in cases than in controls (77% vs 69%, p=0.05). Multivariable analysis shows that the A allele is associated with increased likelihood of CD and there is a dose-response effect (AA vs TT: OR 3.3 p=0.04, AT vs TT: OR 1.9 p=0.8; P for trend=0.03). There is a CARD8 association with CD in the Hispanic population. CARD8, like other CARD family proteins, is involved in apoptosis and NFKB activation. The data shows the existence of a genetic basis for alteration in the innate immune response pathway in the pathogenesis of CD.

### Example 4

### TLR-9 and NOD2/CARD15

The inventors evaluated the association of CARD15 and other innate immune genes including TLR-9 with CD in Puerto Ricans and describe possible phenotypic associations within CD patients. Puerto Rican CD patients (n=113) were recruited from the University of Puerto Rico IBD Clinic. Ethnically matched controls (n=107) were recruited from patients' spouse or general population. Three variants in CARD15 gene (SNPs 8, 12, 13) and two variants in TLR 9-(2848 A/G, 1237C/T) were genotyped by TaqMan. These polymorphisms were evaluated for their association with CD as well as disease behavior, location and IBD-related surgery. The presence of at least one CARD15 variant was observed in 18.7% of CD as compared to 9.4% of controls (p=0.049). The presence of any CARD15 mutation was positively associated with small bowel disease (p=0.06) and negatively associated with perianal involvement (4% vs 34.7%, P=0.0001). A allele of TLR9-2848A/G was more frequent in subjects with CD-related surgery than those without surgery (54% vs 35%, p=0.007). CARD15 was found to be more prevalent in Puerto Ricans with CD as compared to ethnically matched controls. The association of variants of both CARD15 and TLR-9 with specific disease behavior or location shows the influence of genetic variants on clinical expression of the disease.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive. One skilled in the art will recognize many methods and materials similar or equivalent to those described herein, which could be used in the practice of the present invention. Indeed, the present invention is in no way limited to the methods and materials described. Furthermore, one of skill in the art would recognize that the invention can be applied to various inflammatory conditions and disorders and autoimmune diseases besides that of inflammatory bowel disease. It will also be readily apparent to one of skill in the art that the invention can be used in conjunction with a variety of phenotypes, such as serological markers, additional genetic variants, biochemical markers, abnormally expressed biological pathways, and variable clinical manifestations.

1. A method for evaluating the likelihood of an individual to have or develop inflammatory bowel disease, comprising:
determining the presence or absence of a first risk variant at the HPS1 locus, the presence or absence of a second risk variant at the CARD8 locus, and the presence or absence of a third risk variant at the TLR-9 locus,
wherein the presence of one or more risk variants is predictive of inflammatory bowel disease.

2. The method of claim 1, wherein the first risk variant at the HPS1 locus comprises SEQ. ID. NO.: 1.

3. The method of claim 1, wherein the second risk variant at the CARD8 locus comprises SEQ. ID. NO.: 16.

4. The method of claim 1, wherein the third risk variant at the TLR-9 locus comprises SEQ. ID. NO.: 18.

5. The method of claim 1, wherein the individual is Puerto Rican.

6. A method of diagnosing susceptibility to inflammatory bowel disease in an individual, comprising:
determining the presence or absence of a risk haplotype at the HPS1 locus in the individual,
wherein the presence of the risk haplotype is diagnostic of susceptibility to inflammatory bowel disease.

7. The method of claim 6, wherein the individual has not been diagnosed with Hermansky-Pudlak Syndrome.

8. The method of claim 6, wherein said risk haplotype at the HPS1 locus comprises haplotype block 3.

9. The method of claim 6, wherein said risk haplotype at the HPS1 locus comprises SEQ. ID. NO.: 1.

10. The method of claim 6, wherein said individual is Puerto Rican.

11. A method of determining a low probability relative to a healthy individual of developing inflammatory bowel disease in an individual, said method comprising:
determining the presence or absence of a protective haplotype at the IRF1 locus,
wherein the presence of the protective haplotype at the IRF1 locus is diagnostic of a low probability relative to a healthy individual of developing inflammatory bowel disease.

12. The method of claim 11, wherein said protective haplotype at the IRF1 locus comprises H3.

13. The method of claim 11, wherein said protective haplotype at the IRF1 locus comprises one or more variant alleles selected from the group consisting of SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13 and SEQ. ID. NO.: 14.

14. The method of claim 11, wherein said individual is Puerto Rican.

15. A method of diagnosing susceptibility to Crohn's Disease in a Puerto Rican individual, comprising:
determining the presence or absence of a risk variant at the CARD8 locus,
wherein the presence of the risk variant at the CARD8 locus is diagnostic of susceptibility to Crohn's Disease.

16. The method of claim 15, wherein the risk variant at the CARD8 locus comprises SEQ. ID. NO.: 16.

17. The method of claim 15, wherein the individual is Puerto Rican.

18. A method of diagnosing susceptibility to Crohn's Disease in an individual, comprising:
determining the presence or absence of a risk variant at the TLR-9 locus,
wherein the presence of the risk variant at the TLR-9 locus is diagnostic of susceptibility to Crohn's Disease.

19. The method of claim 18, wherein the risk variant at the TLR-9 locus comprises SEQ. ID. NO.: 18.

20. The method of claim 18, wherein the individual is Puerto Rican.

21. A method of treating a non-Hermansky Pudlak form of inflammatory bowel disease in an individual, comprising:
determining the presence of haplotype block 3 at the HPS1 locus to diagnose the non-Hermansky Pudlak form of inflammatory bowel disease; and
treating the non-Hermansky Pudlak form of inflammatory bowel disease.

22. The method of claim 21, wherein the individual is Puerto Rican.

23. A method of treating Crohn's Disease in an individual, comprising:
determining the presence of a risk variant at the CARD8 locus and/or TLR-9 locus; and
treating the Crohn's Disease.

24. The method of claim 23, wherein the individual is Puerto Rican.
